(19) United States
(12) Patent Application Publication
(10) Pub. No.: US 2024/0105072 A1
(43) Pub. Date: Mar. 28, 2024

(54) ANALYSIS APPARATUS, ANALYSIS METHOD, AND NON-TRANSITORY COMPUTER-READABLE MEDIUM

(71) Applicant: NEC Corporation, Minato-ku, Tokyo (JP)

(72) Inventors: Shin Norieda, Tokyo (JP); Hiroo HARADA, Tokyo (JP); Haruki MIZUTANI, Tokyo (JP); Hirotaka MAESHIMA, Tokyo (JP); Masami SAKAGUCHI, Tokyo (JP)

(21) Appl. No.: 18/276,188

(22) PCT Filed: Jan. 14, 2022
(86) PCT No.: PCT/JP2022/001196

§ 371 Date: Aug. 7, 2023

(30) Foreign Application Priority Data
Feb. 25, 2021 (JP) 2021-029036

(51) Int. Cl. G09B 5/08; A61B 5/16; G06V 40/16; G09B 7/00

(52) U.S. Cl. CPC G09B 5/08; A61B 5/165; G06V 40/174; G09B 7/00; G06V 2201/03

(57) ABSTRACT

Provided is an analysis apparatus or the like capable of appropriately determining emotions of a learner or an examinee in online learning or an online examination. An analysis apparatus includes: an emotion data acquisition unit that acquires emotion data regarding learning of each learner, the emotion data being obtained by performing emotion analysis on face image data of a plurality of learners in online learning; and an analysis data generation unit that aggregates emotion data regarding the plurality of learners, compares the emotion data of the plurality of learners, and generates analysis data in which the emotion data of one or more learners is identified.

### TECHNICAL FIELD

The present invention relates to an analysis apparatus, an analysis method, and an analysis program.

### BACKGROUND ART

With advances in information communication technologies, use of online learning and online examinations is expanding. Technologies for analyzing situations (for example, the degree of concentration) of learners during online learning have been proposed.

For example, a learning support apparatus described in Patent Literature 1 includes: first output means for outputting learning information to be provided to a learner; collection means for collecting state information indicating a state of the learner; detection means for detecting the degree of concentration of the learner based on the state information; and forming means for forming encouragement information when it is detected that the degree of concentration of the learner has decreased. The acquisition of the information indicating the learning state of the user is performed by a camera unit, an image recognition processing unit, a microphone, a voice input processing unit, and a voice recognition processing unit. For example, the voice recognition processing unit analyzes voice data and acquires information regarding an emotion of a user indicated by the voice data. Information regarding an expression of the user, information regarding a motion, and information regarding emotions are main information of information indicating a state of the user, and a correct answer rate and a response in accordance with a response input to a test by the user or the like are also considered.

Patent Literature 2 discloses an information processing apparatus including a motion history acquisition unit that acquires motion history information indicating a motion of a learner on a learner terminal, a degree-of-concentration estimation unit that estimates the degree of concentration of the learner based on the motion history information, and a display control unit that changes display on a manager terminal used by a manager of the learner in accordance with information indicating the degree of concentration.

Furthermore, as described in Patent Literature 3, a learning system that provides a lecture including video and audio includes: a concentration determination unit 11 that determines whether a participant is concentrating on the lecture based on an image acquired to capture an image of the participant who participates in the lecture, and records concentration determination result information based on a result of the determination; an understanding determination unit that determines whether the participant understands content of the lecture based on the image and the concentration determination result information and records understanding determination result information based on the result of the determination; and a lecture control unit that controls the lecture based on the concentration determination result information and the understanding determination result information.

### CITATION LIST

### Patent Literature

Patent Literature 1: Japanese Unexamined Patent Application Publication No. 2013-097311
Patent Literature 2: Japanese Unexamined Patent Application Publication No. 2013-242434
Patent Literature 3: Japanese Patent No. 6636670

### SUMMARY OF INVENTION

### Technical Problem

Since the e-learning technique related to the above-described Patent Literature 1 is a form in which a learner learns by himself or herself, it is difficult to appropriately determine a decrease in the degree of concentration. As a result, if the learner whose degree of concentration does not decrease is encouraged, willingness of the learner is actually reduced in some cases. Similarly, even with the techniques according to Patent Literatures 2 and 3, emotions of the learner and the examinee cannot be appropriately determined.

The present disclosure has been made in view of such problems, and an object of the present disclosure is to provide an analysis apparatus or the like capable of appropriately determining emotions of a learner and an examinee in online learning or an online examination.

### Solution to Problem

According to an example embodiment of the present disclosure, an analysis apparatus includes: an emotion data acquisition unit that acquires emotion data regarding learning of each learner, the emotion data being obtained by performing emotion analysis on face image data of a plurality of learners in online learning; and an analysis data generation unit that aggregates emotion data regarding the plurality of learners, compares the emotion data of the plurality of learners, and generates analysis data in which the emotion data of one or more learners is identified based on a comparison result.

According to another example embodiment of the present disclosure, an analysis includes: acquiring emotion data regarding learning of each learner, the emotion data being obtained by performing emotion analysis on face image data of a plurality of learners in online learning; and aggregating emotion data regarding the plurality of learners, comparing the emotion data of the plurality of learners, and generating analysis data in which the emotion data of one or more learners is identified based on a comparison result.

According to still another example embodiment of the present disclosure, an analysis program causes a computer to perform: acquiring emotion data regarding learning of each learner, the emotion data being obtained by performing emotion analysis on face image data of a plurality of learners in online learning; and aggregating emotion data regarding the plurality of learners, comparing the emotion data of the plurality of learners, and generating analysis data in which the emotion data of one or more learners is identified based on a comparison result.

### Advantageous Effects of Invention

According to the present disclosure, it is possible to provide an analysis apparatus, an analysis method, and an analysis program capable of appropriately determining emotions of a learner and an examinee in online learning or an online examination.

### BRIEF DESCRIPTION OF DRAWINGS

FIG. 1 is a block diagram illustrating a configuration of an analysis apparatus according to a first example embodiment.

FIG. 2 is a flowchart illustrating an analysis method according to the first example embodiment.

FIG. 3 is a block diagram illustrating a configuration of an analysis apparatus according to a second example embodiment.

FIG. 4 is a flowchart illustrating an analysis method according to a second example embodiment.

FIG. 5 is a block diagram illustrating a configuration of an analysis system according to a third example embodiment.

FIG. 6 is a block diagram illustrating a configuration of an analysis apparatus according to the third example embodiment.

FIG. 7 is a diagram illustrating an example of data processed by an analysis data generation unit.

FIG. 8 illustrates an example of a distribution for specific emotion data calculated from emotion data of a plurality of learners.

FIG. 9 is a block diagram illustrating a configuration of an emotion data generation apparatus according to the third example embodiment.

FIG. 10 is a flowchart illustrating an analysis method according to the third example embodiment.

FIG. 11 is a diagram illustrating an example of analysis data.

FIG. 12 is a diagram illustrating an example of message data.

FIG. 13 is a diagram illustrating a display example of an analysis result.

### EXAMPLE EMBODIMENT

Hereinafter, example embodiments of the present disclosure will be described in detail with reference to the drawings. In the drawings, the same or corresponding elements are denoted by the same reference numerals, and repeated description is omitted as necessary for clear description.

### First Example Embodiment

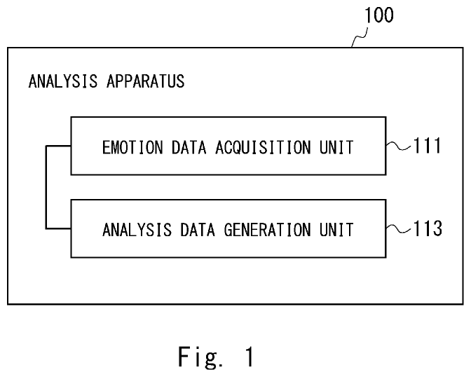

FIG. 1 is a block diagram illustrating a configuration of an analysis apparatus 100 according to a first example embodiment.

The analysis apparatus 100 is implemented by an information processing apparatus such as a computer that includes a processor and a memory. The analysis apparatus 100 is used to analyze an emotion of a learner in learning such as online learning or an online examination.

The analysis apparatus 100 includes: an emotion data acquisition unit 111 that acquires emotion data regarding learning of each of a plurality of learners subjected to emotion analysis on face image data of the plurality of learners in online learning; and an analysis data generation unit 113 that aggregates the emotion data regarding the plurality of learners, compares the emotion data of the plurality of learners, and generates analysis data for identifying the emotion data of one or more learners.

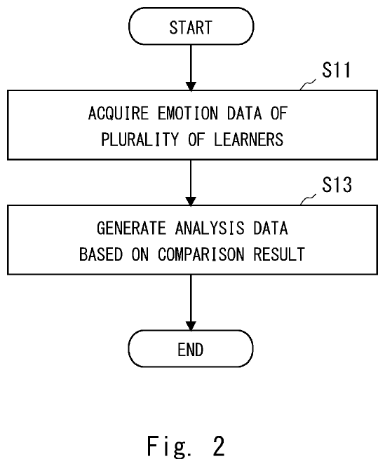

FIG. 2 is a flowchart illustrating an analysis method according to the first example embodiment. The flowchart illustrated in FIG. 2 starts, for example, when the analysis apparatus 100 receives a signal indicating start of learning from a learning management apparatus.

Emotion data regarding learning of each learner in which emotion analysis has been executed on face image data of a plurality of learners in the online learning is acquired (step S11). Emotion data regarding a plurality of learners is aggregated, the emotion data of the plurality of learners is compared, and the emotion data of one or more learners is identified based on a comparison result (step S13).

In the analysis apparatus and the analysis method according to the first example embodiment described above, it is possible to appropriately determine emotions of learners and examinees in online learning and an online examination.

### Second Example Embodiment

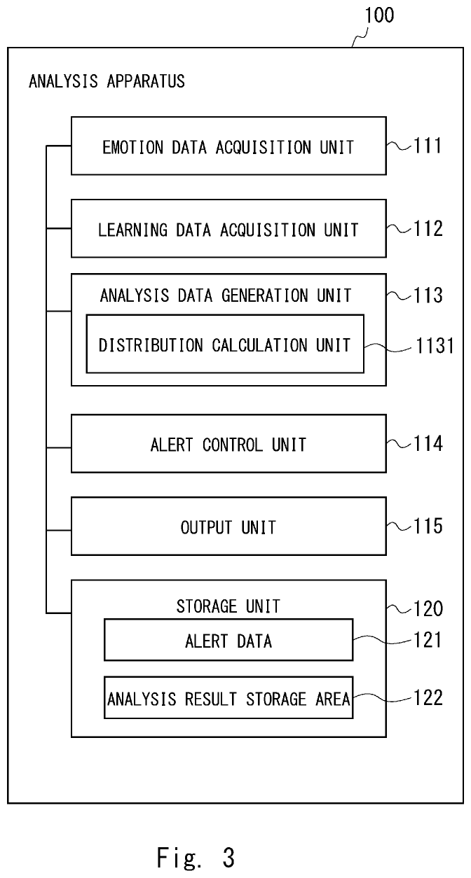

FIG. 3 is a block diagram illustrating a configuration of an analysis apparatus 100 according to a second example embodiment. The analysis apparatus 100 acquires emotion data from face image information of a learner (for example, a learner or an examinee) participating in online learning or an online examination, generates analysis data regarding the online learning or the online examination from the acquired emotion data, and outputs the generated analysis data to a predetermined terminal or the like.

In the example embodiment, the online learning is learning performed using a plurality of learning terminals communicably connected to each other via a communication line. The online learning may have a format in which a class video is delivered in real time or a format in which a class video is delivered on demand. The number of learning terminals is not limited, but may be, for example, the number of students belonging to one class of a school (for example, 20 or 30 students), the number of students corresponding to the first grade of a school (for example, 100 students), the number of examinees of a qualification examination (for example, 3000 examinees), or the like. The online learning used in the present specification includes not only an online class (also referred to as remote joint class) conducted at school, a cram school, or the like, but also an online examination (also referred to as remote joint examination) such as an entrance examination, an employment examination, a selection examination, or a term examination of a school or the like. A learning terminal used for the online learning is, for example, a personal computer, a smartphone, a tablet terminal, a mobile phone with a camera, or the like. The learning terminal is not limited to the above as long as the learning terminal is an apparatus that has a camera that captures an image of a learner, a microphone that collects speeches of a learner, and a communication function of transmitting and receiving image data and audio data. In the following description, the online learning is simply referred to as “learning” in some cases.

In the example embodiment, a learner of online learning is a person who is performing online learning with a learning terminal. Examples of the manager of learning include an organizer of learning, a teacher of learning, and a supervisor of an examination. In the example embodiment, it is assumed that the learner participates in learning in a state in which a face image of the learner can be captured by a camera built in or connected to the learning terminal.

The analysis apparatus 100 is communicably connected to an emotion data generation apparatus that generates emotion data from a face image or the like of a learner in online learning and a learning operation apparatus that operates the learning. The analysis apparatus 100 is communicably connected to a terminal (a manager terminal) of a manager using the analysis apparatus 100. The analysis apparatus 100 mainly includes an emotion data acquisition unit 111, a learning data acquisition unit 112, an analysis data generation unit 113, an alert control unit 114, an output unit 115, and a storage unit 120.

The emotion data acquisition unit 111 acquires emotion data from the emotion data generation apparatus. The emotion data generation apparatus generates emotion data from the face image data of the learner in the online learning, and supplies the generated emotion data to the analysis apparatus 100. The emotion data is data serving as an index indicating an emotion of each learner. The emotion data acquisition unit 111 itself may have a function of generating emotion data.

The emotion data includes, for example, a plurality of items such as a degree of concentration, a degree of confusion, a degree of happiness, anxiety, and surprise. That is, the emotion data indicates how much a learner feels these emotions for each of the above-described items. The emotion data acquired by the emotion data acquisition unit 111 involves time data. The emotion data generation apparatus generates emotion data for each predetermined period of time (for example, one second). The emotion data acquisition unit 111 acquires emotion data for each predetermined time throughout a learning progress time. When the emotion data is acquired, the emotion data acquisition unit 111 supplies the acquired emotion data to the analysis data generation unit 113.

The learning data acquisition unit 112 acquires learning data from a learning management apparatus. The learning management apparatus is, for example, a server apparatus to which each learner is communicably connected through a learning terminal. The learning management apparatus may be included in the learning terminal used by the learner. The learning data is data regarding learning involving time data. More specifically, the learning data includes a start time and an end time of the learning. The learning data includes a time of a break taken during a class.

The learning data acquisition unit 112 acquires learning data including attribute data of learning. The attribute data of the learning can include, for example, information indicating a type of learning such as an online class or an online examination (more specifically, for example, a selection examination, a term examination, and the like). The attribute data of the learning may include information regarding a school to which the learner belongs. The attribute data of the learning may include information regarding a subject of the learning and a purpose of the learning. The learning data acquisition unit 112 supplies the acquired learning data to the analysis data generation unit 113 and the alert control unit 114.

The analysis data generation unit 113 generates analysis data for the learning from the received emotion data, learning data, and data indicating a chapter. The analysis data is data derived from the emotion data and is data extracted or calculated from items indicating a plurality of emotions. The analysis data is preferably an index useful for management of the learning. For example, the analysis data may include the degree of concentration on the learning and the degree of understanding. In this way, the analysis data generation unit 113 generates analysis data corresponding to a plurality of preset analysis items. Accordingly, the analysis apparatus 100 can generate analysis data from a plurality of viewpoints at which the learning is efficiently performed. The analysis data generation unit 113 can generate analysis data for a plurality of learners.

The analysis data generation unit 113 can generate analysis data (for example, transitions of the degree of concentration, anxiety, and the degree of understanding for the learning data) of specific learners by comparing the learning data with emotion data of the specific learners. For example, it is possible to analyze that the degree of concentration of certain learners is reduced for a specific scene in class. However, with only the analysis data of one learner, it is not possible to distinguish whether a problem is a problem of an individual learner or a problem of content of the class. Accordingly, the analysis data generation unit according to the example embodiment can aggregate emotion data of a plurality of learners and statistically process a large amount of data.

The analysis data generation unit 113 further includes a distribution calculation unit 1131. The distribution calculation unit 1131 calculates a distribution of specific analysis data from specific analysis data (for example, the degree of concentration) of each learner (that is, from the aggregated data,). For example, in a scene of a class, a value exceeding a predetermined threshold (for example, standard deviations σ, 2σ, 3σ, or the like) from an average value is identified from a distribution of specific emotion data (for example, the degree of concentration). Accordingly, it is possible to distinguish whether a problem is an individual problem of a learner or a problem of learning data content (for example, a teaching method of a teacher). For example, in a specific scene in a class, when the degree of concentration of almost all the learners is low, it is determined in some cases that almost all the learners are taking notes. Conversely, when some learners have a significantly high degree of concentration in a scene where the degree of concentration of almost all the learners is low, some of the learners may be determined to be taking an abnormal action (for example, the learners are doing something different from the class.). When the degree of concentration of only some of the learners is significantly low, it is determined that some of the learners cannot keep up with the class in some cases.

Even in the case of an online examination, it is possible to statistically process the emotion data of each examinee when each examinee solves the same problem, and to identify the examinee who takes an abnormal behavior. For example, when the degree of concentration of a certain examinee is significantly low, it may be determined that the certain examinee performs cheating.

The analysis data generation unit 113 may set a method of calculating the analysis data in accordance with the attribute data received from the learning data acquisition unit 112. That is, in this case, the analysis data generation unit 113 selects a method of calculating the analysis data in accordance with the attribute data (for example, an online class, an online examinations, and a subject) received from the learning data acquisition unit 112. Accordingly, the analysis apparatus 100 can calculate analysis data in accordance with learning attributes. When the analysis data is generated, the analysis data generation unit 113 supplies the generated analysis data to the alert control unit 114.

The alert control unit 114 receives the analysis data from the analysis data generation unit 113 and reads alert data 121 from the storage unit 120. The alert control unit 114 receives the learning data from the learning data acquisition unit 112. Then, the alert control unit 114 selects a corresponding message from the received data and generates an analysis result including a selected alert. The output unit 115 outputs the alert to the learning terminals or the manager terminal (also referred to as an alert generation unit). The analysis result includes at least analysis data for learning and an alert corresponding to the analysis data. The alert control unit 114 causes the storage unit 120 to store the analysis result so that the analysis result can be output.

For example, when the concentration degree of one or more students is significantly low and greatly deviates from the average value from the analysis data (for example, distribution data of the degree of concentration), an alert indicating “the degree of concentration has decreased” can be extracted and notified of to the system (for example, a learning terminal used by the student or a manager terminal used by a teacher of the student.). Accordingly, the student who has been notified concentrates on the class. Alternatively, the teacher who has been notified can make a contact with a corresponding student (for example, a chat, a mail, a voice call, or the like) to motivate the student.

Alternatively, in another example, for example, when the degree of concentration of one or more students is remarkably high and significantly deviates from the average value although the degree of concentration of other students is decreased (that is, the other students take notes) from the analysis data (for example, distribution data of the degree of concentration), an alert indicating “there is a possibility of one or more students being doing other things” can be extracted and notified of to the system (for example, a learning terminals used by the students or the manager terminal used by the teacher of the student).

Alternatively, in still another example, for example, an alert indicating “there is a possibility of the student not being able to keep up with the class” may be extracted from the analysis data (for example, analysis data of the degree of anxiety) to students feeling anxiety more than other students.

Alternatively, in still another example, for example, during the online examination, when the concentration degree of one or more students is significantly lower than that of the other students and significantly deviates from the average value, an alert indicating “there is a possibility of cheating” can be extracted and notified of to the system (for example, the learning terminals used by the students or a manager terminal used by the teacher of the students).

The output unit 115 outputs an analysis result stored in the storage unit 120 to the learning terminals or the manager terminal. The output unit 115 outputs the alert to the manager terminal or the learning terminals (also referred to as an alert generation unit). The manager (for example, an organizer, a teacher, an examination supervisor, and the like of the learning) who uses the analysis apparatus 100 can recognize which kind of emotion the learner has toward learning content, content of examination questions, a statement of the teacher or another student, or the like by perceiving the analysis result received by the manager terminal. The manager using the analysis apparatus 100 can recognize which action the manager should take for the next learning by perceiving an alert or an advice included in the analysis result. Therefore, the manager can perceive, from the received analysis data, attentions or the like for learning to be held later.

The storage unit 120 is a storage device including a non-volatile memory such as a solid state drive (SSD) or a flash memory. The storage unit 120 includes alert data 121 and an analysis result storage area 122. The alert data 121 is data in which an alert pattern to be presented to the manager is associated with learning data. The analysis result storage area 122 is an area where the analysis result generated by the alert control unit 114 is stored.

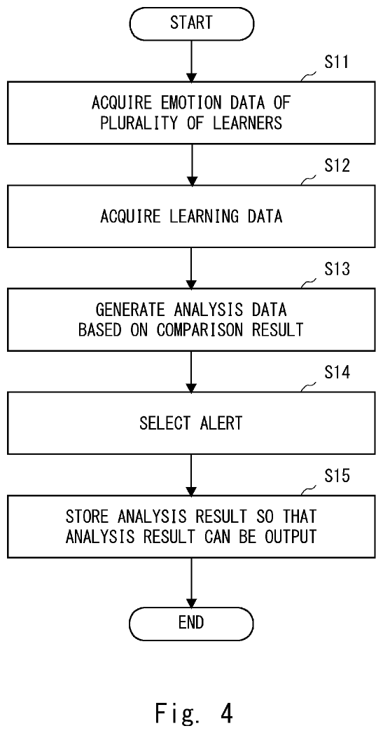

Next, a process of the analysis apparatus 100 according to the first example embodiment will be described with reference to FIG. 4. FIG. 4 is a flowchart illustrating an analysis method according to the second example embodiment. The flowchart illustrated in FIG. 4 starts, for example, when analysis apparatus 100 receives a signal indicating start of the learning from the learning management apparatus.

First, the emotion data acquisition unit 111 acquires emotion data of a plurality of learners from the emotion data generation apparatus (step S11). The emotion data acquisition unit 111 may acquire the generated emotion data whenever the emotion data generation apparatus generates the emotion data, or may collectively acquire the emotion data at a plurality of different times.

Next, the learning data acquisition unit 112 acquires learning data regarding learning involving time data (step S12). The learning data acquisition unit 112 may receive the learning data every predetermined period (for example, 1 minute) or sequentially receive the learning data when there is information to be updated in the learning data. The learning data acquisition unit 112 may receive the learning data after the learning is completed. It is assumed that the plurality of learners learn using the same learning data.

Subsequently, the analysis data generation unit 113 generates analysis data for the learning from the emotion data of the plurality of learners received from the emotion data acquisition unit 111 and the learning data received from the learning data acquisition unit 112 (step S13). The emotion data from the plurality of learners can be relatively compared to be able to generate, for example, analysis data in which a learner who takes an abnormal behavior is identified.

Subsequently, the alert control unit 114 selects an alert corresponding to the analysis data from the alert data 121 of the storage unit 120 (step S14). Further, the alert control unit 114 stores the analysis result including the selected alert in the analysis result storage area 122 of the storage unit 120 so that the analysis result can be output (step S15).

The processes performed by the analysis apparatus 100 has been described above. Of the above-described processes, the order of steps S11 and S12 does not matter. Steps S11 and S12 may be executed in parallel. Alternatively, steps S11 and S12 may be alternately performed every predetermined period.

As described above, the analysis apparatus 100 according to the first example embodiment acquires the emotion data and the learning data of the learners in the online learning and generates the analysis data for the learning. Then, the analysis apparatus 100 selects an alert corresponding to the analysis data and stores the selected alert so that the alert can be output. Accordingly, the user using the analysis apparatus 100 can grasp the analysis result by the alert corresponding to the analysis data in the online learning. Thus, according to the example embodiment, it is possible to provide the analysis apparatus, the analysis method, the analysis system, and the analysis program capable of appropriately determining emotions of the learners and the examinee in the online learning or the online examination.

The analysis apparatus 100 includes a processor and a storage device as a configuration (not illustrated). The storage device included in the analysis apparatus 100 includes a storage device including a non-volatile memory such as a flash memory or an SSD. The storage device included in the analysis apparatus 100 stores a computer program (hereinafter also simply referred to as a program) executing the analysis method according to the example embodiment. The processor also reads a computer program from the storage device to the memory and executes the program.

Each configuration of the analysis apparatus 100 may be implemented with dedicated hardware. Some or all of the constituents may be implemented by general-purpose or dedicated circuitry, a processor, or the like, or a combination thereof. These units may be configured with a single chip or may be configured with a plurality of chips connected via a bus. Some or all of the constituents of each apparatus may be implemented in a combination of the above-described circuit or the like and a program. As the processor, a central processing unit (CPU), a graphics processing unit (GPU), a field-programmable gate array (FPGA), or the like can be used.

When some or all of the constituents of the analysis apparatus 100 are implemented with a plurality of computation apparatuses, circuits, and the like, the plurality of computation apparatuses, circuits, and the like may be disposed in a centralized manner or in a distributed manner. For example, the computation apparatuses, the circuits, and the like may be implemented in a form in which each of them is connected via a communication network, such as a client server system or a cloud computing system. The function of the analysis apparatus 100 may be provided in software as a service (SaaS) format.

### Third Example Embodiment

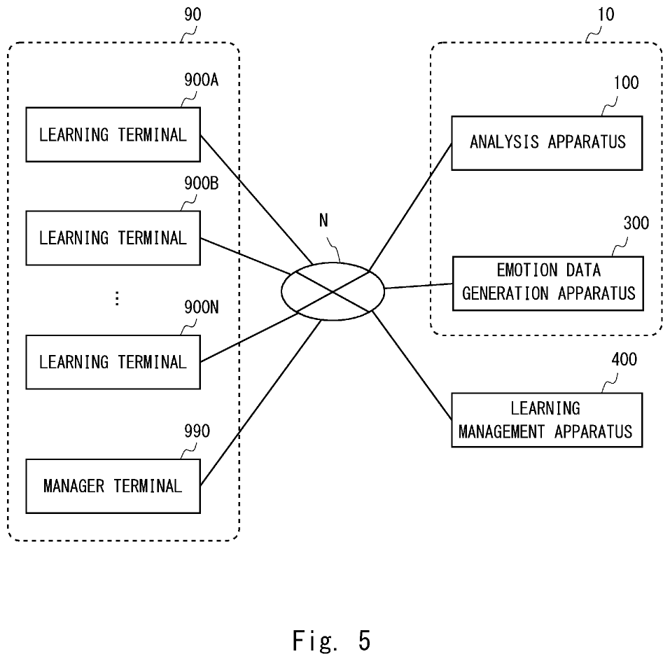

FIG. 5 is a block diagram illustrating a configuration of an analysis system according to a third example embodiment. An analysis system 10 illustrated in FIG. 5 includes an analysis apparatus 100 and an emotion data generation apparatus 300. The analysis apparatus 100 and the emotion data generation apparatus 300 are communicably connected to each other via a network N. The analysis system 10 is communicably connected to a learning management apparatus 400 via the network N. The learning management apparatus 400 is connected to a learning terminal group 90 via the network N to operate the online learning. The learning terminal group 90 includes a plurality of learning terminals (900A, 900B, . . . , 900N) and a manager terminal 990.

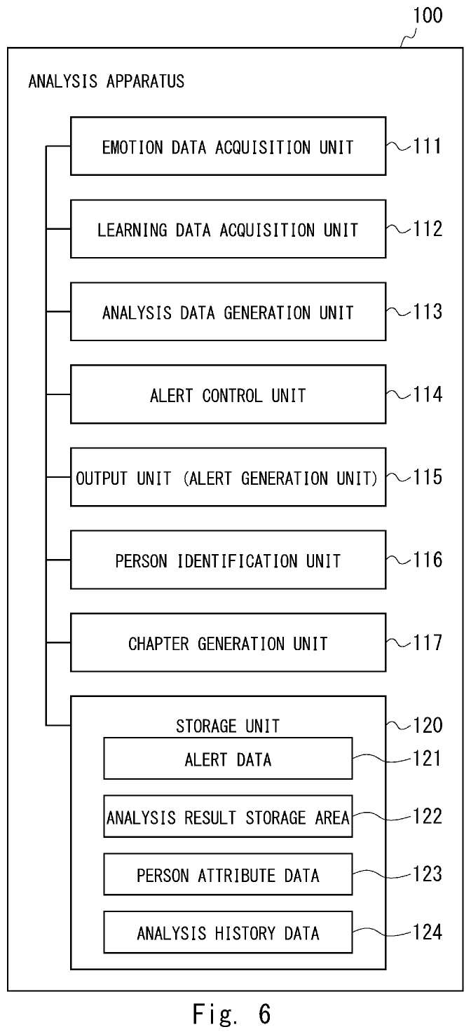

Next, an analysis apparatus according to a third example embodiment will be described with reference to FIG. 6. FIG. 6 is a block diagram illustrating a configuration of the analysis apparatus 100 according to the third example embodiment. The analysis apparatus 100 according to the second example embodiment is different from the analysis apparatus 100 according to the second example embodiment in that a person identification unit 116 and a chapter generation unit 117 are included. Hereinafter, each configuration of the analysis apparatus 100 according to the example embodiment will be described, including different points from the analysis apparatus 100 according to the first example embodiment.

The emotion data acquisition unit 111 according to the example embodiment acquires emotion data in which a plurality of indices indicating emotional states are indicated by numerical values. The analysis data generation unit 113 generates analysis data by calculating statistical values of the emotion data during a predetermined period. The emotion data acquisition unit 111 can acquire emotion data including identification information of the learning terminal. That is, in this case, the emotion data acquisition unit 111 can acquire the respective emotion data of the learners in a distinguishable manner.

The emotion data acquisition unit 111 can acquire the emotion data involving time data related to the learning. Since the emotion data involves the time data, the emotion data acquisition unit 111 can acquire, for example, emotion data for generating analysis data for each chapter, as will be described below.

The learning data acquisition unit 112 acquires learning data from the learning management apparatus 400 that manages the learning. The learning data acquisition unit 112 acquires learning data including attribute data of learning. The learning data acquisition unit 112 can acquire face image data of the learners from the learning management apparatus 400. Furthermore, the learning data acquisition unit 112 can acquire the learning data including data for identifying speakers in the learning.

The learning data acquisition unit 112 may acquire learning data including data related to screen sharing in the learning. In this case, the learning data can include, for example, a switching time of an authority to operate the shared screen shared by the learners (the manager of the shared screen) and the switching time of the speeches of the learners. The learning data acquisition unit 112 may acquire the learning data including screen data shared in the learning. In this case, the learning data may include a time such as page transmission in the shared screen or a change in a display image. Furthermore, the learning data can include data indicating what each of the above-described times indicates. The learning data acquisition unit 112 supplies the acquired face image data to the person identification unit 116.

The learning data acquisition unit 112 can acquire learning data involving time data. By acquiring the learning data involving time data, the learning data acquisition unit 112 can acquire the learning data for generating analysis data for each chapter, for example, as will be described below.

The analysis data generation unit 113 generates analysis data for the learning from the received emotion data and learning data. The analysis data generation unit 113 can generate analysis data for the learning for each chapter from data indicating a chapter received from the chapter generation unit 117.

The analysis data generation unit 113 can generate analysis data by distinguishing students and a teacher from each other. In this case, the analysis data generation unit 113 acquires the emotion data and the learning data so that the learners can be distinguished. At this time, the learning data includes data (for example, which class the student is, whether a speaker is a teacher or a student, and the like.) indicating which learner is a speaker in a class. Accordingly, the analysis data generation unit 113 can generate the pieces of analysis data after distinguishing the emotion data of speakers and the emotion data of non-speakers from each other. The analysis data generation unit 113 supplies the analysis data generated as described above to the alert control unit 114.

The analysis data generation unit 113 can generate analysis data including a relative comparison result corresponding to the learning attribute data from the learning attribute data and the analysis history data 124 stored in the storage unit 120. That is, the analysis data generation unit 113 extracts the analysis data having the attribute data corresponding to the attribute data included in the learning data related to the analysis from the analysis history data 124 and generates the relative comparison result.

The analysis data generation unit 113 can also generate analysis data based on a relative comparison of emotion data for a plurality of students. As described above, the analysis data generation unit 113 can calculate a distribution of the emotion data of the plurality of learners and identify emotion data exceeding a predetermined threshold (for example, standard deviations σ, 2σ, 3σ, or the like) from the average value. The analysis data generation unit 113 may preferentially extract the latest data from the analysis history data 124. The analysis data generation unit 113 may calculate statistical values of scores of the analysis data in the corresponding attribute data from the analysis history data 124 and then perform relative comparison.

When data indicating a chapter is generated for the learning, the analysis data generation unit 113 can generate analysis data for the learning for each chapter. Accordingly, the analysis apparatus 100 can generate analysis data for each chapter and supply an alert corresponding to the generated analysis data.

When the analysis data received from the analysis data generation unit 113 includes a plurality of analysis items, the alert control unit 114 can select an alert based on the analysis item. For example, when the analysis data includes scores for the analysis items such as the degree of concentration, the degree of empathy, and the degree of understanding, the alert control unit 114 can select an alert for the score of the degree of concentration, an alert for the degree of empathy, and an alert for the degree of understanding. Accordingly, the analysis apparatus 100 can supply a detailed alert or an advice to the user.

When the analysis data exceeds a range of a preset threshold, the alert control unit 114 can select, as an alert, an advice for causing the analysis data to fall within the range of the threshold. For example, it is assumed that the analysis data generation unit 113 generates a score of “the degree of understanding” which is an analysis item with a numerical value between 0 and 100, and that the larger the numerical value is, the higher the degree of understanding of the learner is. It is assumed that the alert control unit 114 sets a threshold 50 for the degree of understanding. In this case, when the analysis data of the degree of understanding is less than 50, the alert control unit 114 selects an advice for making the score higher than 50 from the alerts stored in the alert data 121. For example, in this case, the alert data 121 stores an alert indicating “Improve the degree of understanding” and the alert control unit 114 selects this alert. In such a configuration, the analysis apparatus 100 can supply the manager or the learner with an advice for performing effective learning.

When analysis data in which a speaker and a non-speaker are distinguished from each other is received from the analysis data generation unit 113, the alert control unit 114 generates an analysis result including an alert for the speaker from the received analysis data and stores the analysis result in the storage unit 120. In such a configuration, the analysis apparatus 100 selects an alert for each of the analysis data of the speaker and the analysis data of the non-speaker. Therefore, the user can grasp the analysis data and the alert from the viewpoint of each of the speaker and the non-speaker.

When the analysis data is generated for each chapter, the alert control unit 114 selects an alert for each of the generated analysis data for each chapter. Accordingly, the analysis apparatus 100 can supply an alert or an advice for each chapter.

The person identification unit 116 can have a function of extracting face feature information of a person related to the face image from the face image data and estimating a division to which the person belongs in accordance with to the extracted information. The division to which the person belongs indicates a feature or an attribute of the person, for example, an age or sex of the person. The person identification unit 116 uses the above-described function to identify a division to which the learner belongs in the face image data received from the learning data acquisition unit 112. The person identification unit 116 supplies data regarding the division of the person to the analysis data generation unit 113.

The person identification unit 116 may identify a division to which the identified learner belongs using person attribute data 123 stored in the storage unit 120. In this case, the person identification unit 116 associates the face feature information extracted from the face image with the person attribute data 123 and identifies the division of the learner corresponding to the face feature information. The division of the learner in this case is, for example, a school to which the learner belongs, a class in the school, or the like. In such a configuration, the analysis apparatus 100 can extract data available for the analysis data while taking privacy of the learner into consideration.

The person identification unit 116 may identify a person related to the face image from the face image data received from the learning data acquisition unit 112. In this case, the person identification unit 116 associates the face feature information extracted from the face image with the person attribute data 123 stored in the storage unit 120, and identifies a learner corresponding to the face feature information. Accordingly, the person identification unit 116 can identify each of the learners. By specifying the learner, the analysis apparatus 100 can generate analysis data associated with the identified learner. Accordingly, the analysis apparatus 100 can perform detailed analysis on the identified learner.

The chapter generation unit 117 generates a chapter for the learning from the learning data received from the learning data acquisition unit 112. The chapter generation unit 117 detects, for example, a time from start of the learning to end of the learning, further detects a time matching a preset condition, and generates data indicating the chapter using each time as a division. The chapter of the learning in the present disclosure is defined by whether a state matching the predetermined condition is maintained in the learning or whether the predetermined condition is changed. The chapter generation unit 117 may generate a chapter based on, for example, data regarding screen sharing. More specifically, the chapter generation unit 117 may generate a chapter in accordance with a timing at which the screen sharing is switched. The chapter generation unit 117 may generate a chapter in accordance with a time at which an owner of the shared screen in the screen sharing is switched. The chapter generation unit 117 supplies data indicating the generated chapter to the analysis data generation unit 113.

The storage unit 120 is a storage apparatus including a non-volatile memory such as an SSD or a flash memory. The storage unit 120 stores person attribute data 123 and analysis history data 124 in addition to the alert data 121 and the analysis result storage area 122.

The person attribute data 123 is data in which face feature information of a person is associated with information regarding a division and an attribute of the person. The information regarding the division and attribute of the person is, for example, a name, a sex, an age, an affiliated school, an affiliated company, or a type of occupation of the person, but is not limited thereto.

The analysis history data 124 is analysis data related to analysis performed by the analysis apparatus 100 in the past, that is, analysis data generated by the analysis data generation unit 113 of the analysis apparatus 100 in the past. In addition to the above-described data, the storage unit 120 stores, for example, a program executing an analysis method according to the example embodiment.

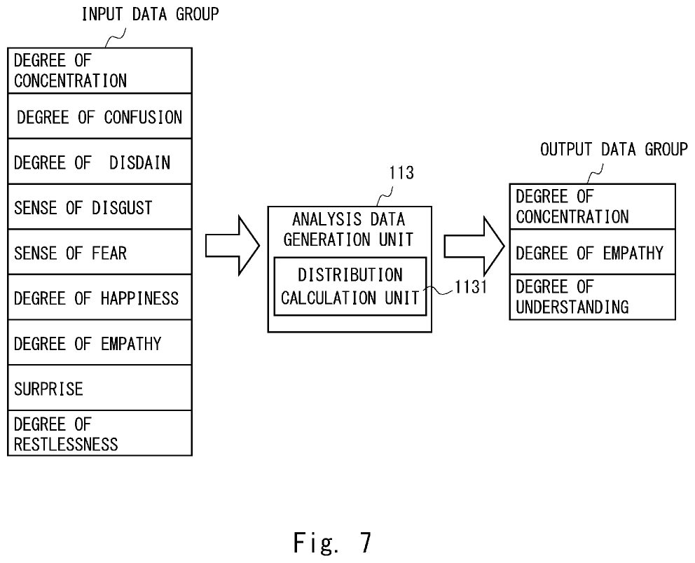

The analysis data generation unit 113 will be further described with reference to FIG. 7. FIG. 7 is a diagram illustrating an example of data processed by the analysis data generation unit. FIG. 7 illustrates an input data group received by the analysis data generation unit 113 and an output data group output by the analysis data generation unit 113. The analysis data generation unit 113 receives emotion data as an input data group from the emotion data generation apparatus 300. The input data group includes, for example, indices related to the degree of concentration, the degree of confusion, the degree of disdain, a sense of disgust, a sense of fear, the degree of happiness, the degree of anxiety, the degree of empathy, a surprise, and the degree of restlessness. These indices are indicated, for example, by numerical values from 0 to 100. The index illustrated here indicates that, for example, the larger the value is, the greater a reaction of the learner to the emotion is. The emotion data of the input data group generated from face image data using a known video processing technology may be acquired or may be generated and acquired by another method.

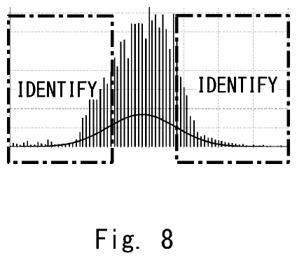

Further, the analysis data generation unit 113 includes a distribution calculation unit 1131. The distribution calculation unit 1131 calculates a distribution for specific emotion data from the emotion data of a plurality of learners (for example, the number of learners is 20 or more, 30 or more, or 100 or more). Here, the number of plurality of learners can be a number corresponding to one class or a number corresponding to the first grade. FIG. 8 illustrates an example of the distribution for the specific emotion data calculated from the emotion data of the plurality of learners. In FIG. 8, the horizontal axis represents the degree of concentration and the vertical axis represents the number of students. The distribution calculation unit 1131 can identify a range (deviation value) exceeding a predetermined threshold (for example, a standard deviation σ, 2σ, 3σ, or the like) from the average value. The distribution calculation unit 1131 can identify an upper limit range (exceeding, for example, the standard deviation σ), a lower limit range (less than, for example, a standard deviation −σ.) or both the upper limit range and the lower limit range. For example, analysis data in which students having the low degree of concentration falling within the lower limit range of the distribution are identified is generated in some cases. In this way, by statistically analyzing the emotion data of the plurality of learners, it is possible to identify a learner who takes an abnormal action. The abnormal behavior is, for example, but are not limited to, poor concentration, a failure to keep up with a class, and a suspicion of a cheating behavior.

When the above-described input data group is received, the analysis data generation unit 113 performs a preset process and generates an output data group using the input data group. The output data group is data that is referred to by a user who uses the analysis system 10 to efficiently perform the learning. Examples of the output data group include the degree of concentration, the degree of empathy, and the degree of understanding. The analysis data generation unit 113 extracts a preset index from the input data group. The analysis data generation unit 113 performs a preset calculation process on a value regarding the extracted index. Then, the analysis data generation unit 113 generates the above-described output data group. The degree of concentration indicated as the output data group may be the same as or different from the degree of concentration included in the input data group. Similarly, the degree of empathy indicated as the output data group may be the same as or different from the degree of empathy included in the input data group.

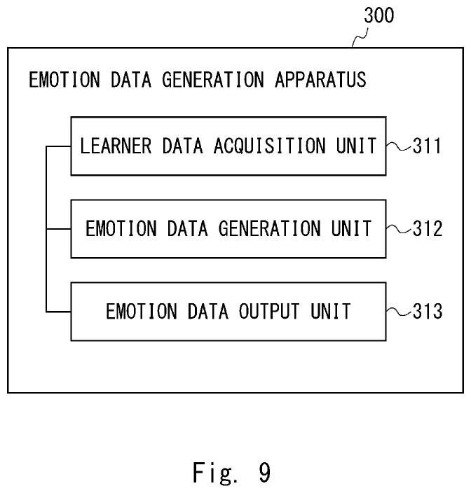

Next, the emotion data generation apparatus 300 will be described with reference to FIG. 9. FIG. 9 is a block diagram illustrating a configuration of an emotion data generation apparatus according to the third example embodiment. The emotion data generation apparatus 300 includes a learner data acquisition unit 311, an emotion data generation unit 312, and an emotion data output unit 313 as main constituents.

The learner data acquisition unit 311 acquires data regarding the learners from the learning management apparatus 400. The data regarding the learners is face image data of the learners captured by the learning terminal. In another example, the data regarding the learners may acquire biological information such as a heart rate and a pulse from a wearable apparatus (for example, a smartwatch) worn by the learner. The emotion data generation unit 312 generates emotion data from the face image data received by the emotion data generation apparatus 300. The emotion data generation unit 312 may additionally generate emotion data from the biometric information. The emotion data output unit 313 outputs the emotion data generated by the emotion data generation unit 312 to the analysis apparatus 100 via the network N. The emotion data generation apparatus 300 generates emotion data by performing predetermined image processing on the face image data of the learners. The predetermined image processing is, for example, extraction of feature points (or features), comparison of the extracted feature points with reference data, a convolution process of image data, a process using training data trained by machine learning, a process using training data by deep learning, and or like. However, a scheme by which the emotion data generation apparatus 300 generates the emotion data is not limited to the above-described processes. The emotion data may be a numerical value that is an index indicating an emotion or may include image data used to generate the emotion data.

The data related to the learner may include data for distinguishing the learners from each other. For example, the data regarding the learners may include identifiers of learning terminals capturing the face image data of the learners. Accordingly, the emotion data generation unit 312 can generate the emotion data in a state in which the learners can be distinguished from each other. Then, the emotion data output unit 313 generates the emotion data corresponding to the learning terminals so that the learning terminals can be distinguished from each other and supplies the emotion data to the emotion data acquisition unit 111.

The emotion data generation apparatus 300 includes a processor and a storage device as a configuration (not illustrated). The storage device included in the emotion data generation apparatus 300 stores a program executing emotion data generation according to the example embodiment. The processor also reads a program from the storage device to the memory and executes the program.

Each configuration of the emotion data generation apparatus 300 may be implemented with dedicated hardware. Some or all of the constituents may be implemented by a general-purpose or dedicated circuit, processor, or the like, or a combination thereof. These units may be configured with a single chip or may be configured with a plurality of chips connected via a bus. Some or all of the constituents of each apparatus may be implemented in a combination of the above-described circuit or the like and a program. As the processor, a CPU, a GPU, an FPGA, or the like can be used.

When some or all of the constituents of the emotion data generation apparatus 300 are implemented by a plurality of computation apparatuses, circuits, and the like, the plurality of computation apparatuses, circuits, and the like may be disposed in a centralized manner or in a distributed manner. For example, the computation apparatuses, the circuits, and the like may be implemented in a form in which each of them is connected via a communication network, such as a client server system or a cloud computing system. The function of the emotion data generation apparatus 300 may be provided in a SaaS format.

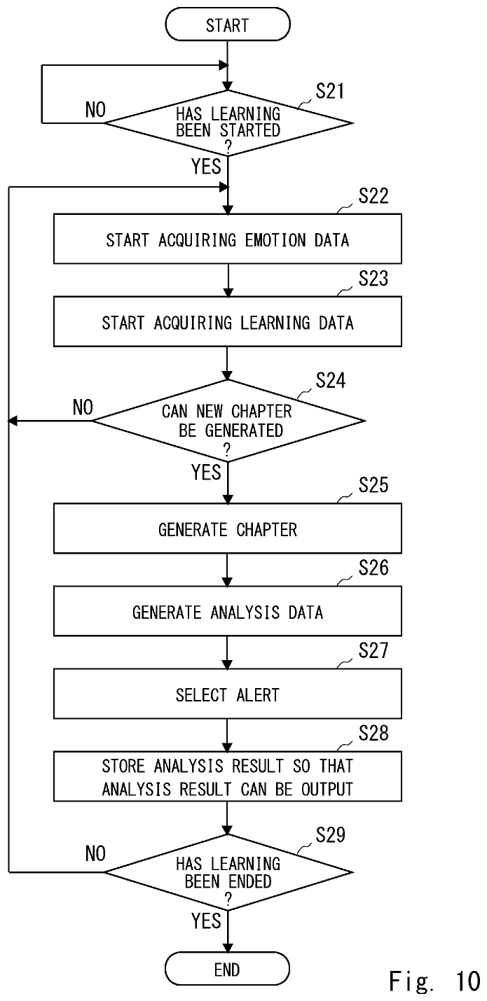

Next, a process executed by the analysis apparatus 100 will be described with reference to FIG. 10. FIG. 10 is a flowchart illustrating an analysis method according to the third example embodiment. The process illustrated in FIG. 10 is different from the process according to the second example embodiment in that analysis data is output whenever a new chapter is generated in the learning.

First, the analysis apparatus 100 determines whether the online learning has been started (step S21). The analysis apparatus 100 determines whether the learning (for example, a class or an examination) has been started by receiving a signal indicating that learning is started from learning management apparatus 400. In a case in which it is determined that the online learning has not been started (NO in step S21), the analysis apparatus 100 repeats step S21. When it is determined that the online learning has been started (YES in step S21), the analysis apparatus 100 proceeds to step S22.

In step S22, the emotion data acquisition unit 111 starts acquiring the emotion data from the emotion data generation apparatus (step S22). The emotion data acquisition unit 111 may acquire the generated emotion data whenever the emotion data generation apparatus generates the emotion data, or may collectively acquire the emotion data at a plurality of different times.

Subsequently, the learning data acquisition unit 112 acquires the learning data regarding learning with time data (step S23). The learning data acquisition unit 112 may receive the learning data every predetermined period (for example, 1 minute) or sequentially receive the learning data when there is information to be updated in the learning data.

Subsequently, the analysis apparatus 100 determines whether a new chapter can be generated from the received learning data (step S24). When it is not determined that the new chapter can be generated (NO in step S24), the analysis apparatus 100 returns to step S22. Conversely, when it is determined that the new chapter can be generated (YES in step S24), the analysis apparatus 100 moves to step S25.

In step S25, the chapter generation unit 117 generates a chapter from the learning data received from the learning data acquisition unit 112 (step S25).

Subsequently, the analysis data generation unit 113 generates analysis data for the newly generated chapter from the emotion data received from the emotion data acquisition unit 111, the learning data received from the learning data acquisition unit 112, the data indicating the chapter received from the chapter generation unit 117, and the data received from the person identification unit 116 (step S26). It is possible to generate analysis data in which a learner taking an abnormal action is identified based on the distribution of the emotion data of the plurality of learners.

Subsequently, the alert control unit 114 selects an alert corresponding to the analysis data from the alert data 121 in the storage unit 120 (step S27). Further, the alert control unit 114 stores the analysis result including the selected alert in the analysis result storage area 122 of the storage unit 120 so that the analysis result can be output (step S28).

Subsequently, the analysis apparatus 100 determines whether the learning has ended (step S29). The analysis apparatus 100 determines whether the learning has ended by receiving a signal indicating that the learning has been completed from the learning management apparatus 400. When it is determined that the learning has not ended (NO in step S29), the analysis apparatus 100 returns to step S22 and continues the process. Conversely, when it is determined that the online learning has ended (YES in step S29), the analysis apparatus 100 ends a series of processes.

The process of the analysis apparatus 100 according to the third example embodiment has been described above. According to the above-described flowchart, the analysis apparatus 100 generates the analysis data for the chapter generated whenever the new chapter is generated in the learning, and can select an alert corresponding to the generated analysis data. Accordingly, the user using the analysis system 10 can effectively proceed with the learning by using an alert or an advice supplied whenever a new chapter is generated in the learning. Alternatively, the manager or the learner can achieve smooth communication between the teacher and the student, for example, by using the alert or the advice supplied whenever a new chapter is generated in the learning.

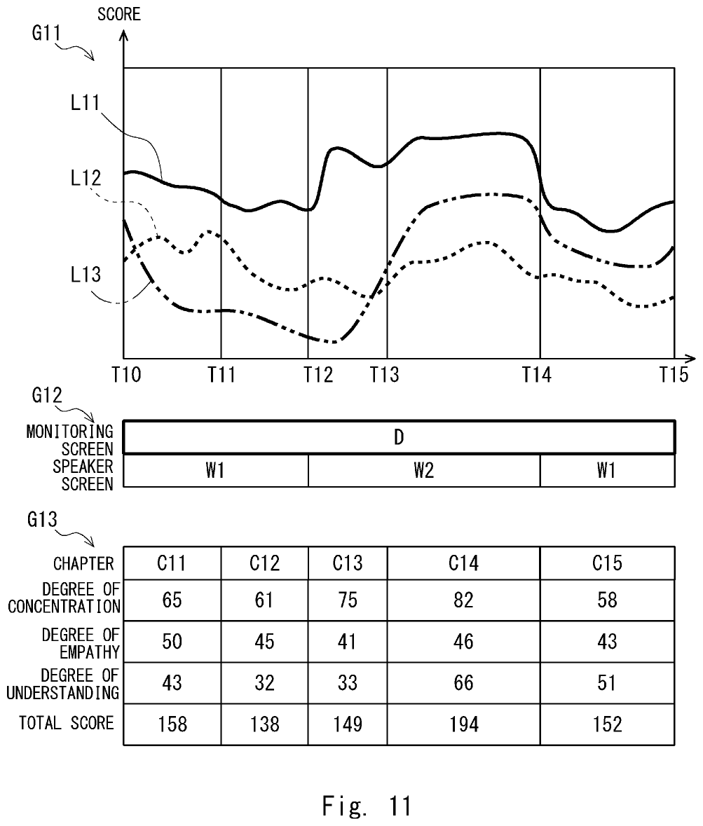

Next, an example of the analysis data generated by the analysis data generation unit 113 will be described with reference to FIG. 11. FIG. 11 is a diagram illustrating an example of the analysis data. In FIG. 11, a graph G11 that chronologically shows the analysis data is illustrated in the upper part. The graph G11 shows transition of the analysis data of all students of a certain class. Learning data G12 corresponding to the time series is illustrated in the middle part. The learning data indicates a monitoring screen on which the face of a student of the class is captured and a speaker screen on which a speaker (mainly a teacher) is captured (the screen can also be switched to a screen on which a textbook, a blackboard, or the like is captured). In the lower part, the analysis data G13 for each chapter corresponding to the learning data is shown.

In the graph G11, the horizontal axis represents time and the vertical axis represents a score of the analysis data. On the horizontal axis, the left end is time T10, the time passes as it goes to the right, and the right end is time T15. Time T10 is a start time of the learning, and time T15 is an end time of the learning. Times T11, T12, T13, and T14 between time T10 and time T15 indicate times corresponding to chapters to be described below.

In the graph G11, first analysis data L11 presented by a solid line, second analysis data L12 represented by a dotted line, and third analysis data L13 represented by a two-dot chain line are plotted. The first analysis data L11 indicates the degree of concentration in the analysis data. The second analysis data L12 indicates the degree of empathy in the analysis data. The third analysis data L13 indicates the degree of understanding in the analysis data.

In the learning data G12, data regarding a learner monitoring screen in class and data regarding a speaker screen are illustrated chronologically. That is, a face image of each student is displayed in the data regarding the learner monitoring screen. A face image of each student may be displayed together on the learner monitoring screen, or a face image of the student may be switched and displayed at predetermined time intervals.

In the learning data G12, data regarding the speaker indicates that a period from time T10 to time T12 is a speaker W1 (for example, a teacher). The data regarding the speaker indicates that a period from time T12 to time T14 is a speaker W2 (for example, a certain student), and a period from time T14 to time T15 is the speaker W1 (for example, the teacher) again.

A relationship between the monitoring screen and the speaker screen (for example, mainly the teacher) in the above-described learning data G12 will be described in chronological order. From time T10 to time T15, a face image of each student is displayed on the monitoring screen. During a period from time T10 at which the learning is started to time T12, the speaker W1 is progressing the learning. During a period from time T10 to time T11, the speaker W1 is displayed on the speaker screen. At time T11, the speaker W1 has switched to display a part of the textbook on the speaker screen. Next, at time T12, the speaker is shifted from the speaker W1 to the speaker W2 (for example, the certain student). The speaker W2 was displayed on the speaker screen from time T12 to time T13. At time T13, the speaker W2 has switched the screen to display a part of the blackboard. Between time T14 and time T15, the speaker W1 shifted from the speaker W2 has been displayed again on the speaker screen.

The relationship between the monitoring screen and the speaker screen in the learning data G12 has been described in the chronological order. As described above, the learning data illustrated in FIG. 11 includes data regarding a period in which the screen data on the monitoring screen is displayed and data regarding the speaker screen indicating who the speaker is. The chapter generation unit 117 generates a chapter in accordance with data regarding the speaker screen among the above-described learning data. The chapter may also be generated at a timing at which the speaker switches to the screen on which the textbook, the blackboard, or the like is captured.

In the analysis data G13, data indicating a chapter corresponding to the above-described learning data and analysis data corresponding to the chapter are illustrated in chronological order. In the example illustrated in FIG. 11, data indicating a chapter corresponds to data regarding the speaker screen in the learning data. That is, a first chapter C11 is a chapter from time T10 at which the speaker W1 is displayed on the speaker screen to time T11. Similarly, a second chapter C12 is a chapter from time T11 at which a part of the textbook is displayed on the speaker screen W1 to time T12. A third chapter C13 is a chapter from time T12 to time T13 at which the speaker W2 is shared on the speaker screen. A fourth chapter C14 is a chapter from time T13 at which a part of the blackboard is displayed on the speaker screen W2 to time T14. A fifth chapter C15 is a chapter from time T14 to time T15 at which the speaker W1 is shared on the speaker screen.

As illustrated in FIG. 11, the analysis data G13 includes analysis data corresponding to each chapter. The analysis data indicates the degree of concentration, the degree of empathy, the degree of understanding, and a total score obtained by summing the degree of concentration, the degree of empathy, and the degree of understanding. In the analysis data G13, for example, as the analysis data corresponding to the chapter C11, the degree of concentration is 65, the degree of empathy is 50, and the degree of understanding is 43. In the total score, 158 is shown as the sum of these scores. Similarly, for example, as the analysis data corresponding to the chapter C12, the degree of concentration is 61, the degree of empathy is 45, the degree of understanding is 32, and the total score is 138.

The analysis data corresponds to data plotted in the graph G11. That is, the analysis data indicated as the analysis data G13 is an average value of the analysis data calculated every predetermined period (for example, 1 minute) in a period of the corresponding chapter.

The example of the analysis data has been described above. In the example illustrated in FIG. 11, the chapter generation unit 117 sets a timing at which the speaker screen in the learning data is switched to a chapter switching timing. A timing at which the speaker switches the screen to the textbook, the blackboard, or the like is also set as the chapter switching timing. Then, the analysis data generation unit 113 calculates analysis data from start of learning to end of learning for each chapter described above. Accordingly, the analysis system 10 can supply the analysis data for each of the displayed speaker screen and other screens.

In the example illustrated in FIG. 11, the analysis system 10 calculates and plots the analysis data every predetermined period as illustrated in the above-described graph G11. Accordingly, the analysis system 10 can show a detailed change in the analysis data in the learning. However, instead of the calculation as illustrated in the graph G11, the analysis data generation unit 113 may first calculate a statistical value (for example, an average value) of the emotion data in the chapter after end of the chapter, and then calculate the analysis data. For example, as illustrated in FIG. 8, the distribution can be calculated in order to relatively compare the emotion data of each learner. A range exceeding a predetermined threshold (for example, the standard deviation σ, 2σ, 3σ, or the like) from the average value can be identified from the distribution. In such a configuration, the analysis system 10 can improve a processing speed of the analysis data.

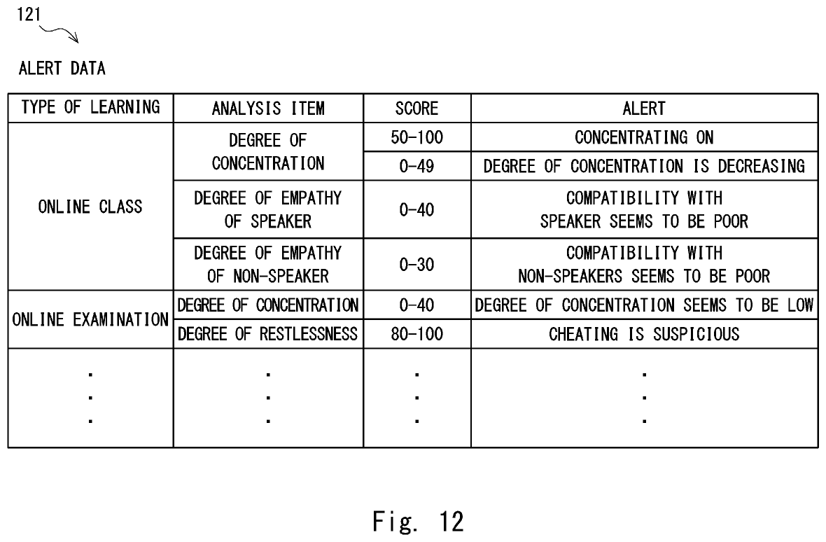

Next, the alert data 121 will be described with reference to FIG. 12. FIG. 12 is a diagram illustrating an example of the alert data. A table illustrated in FIG. 12 illustrates a type of learning, an analysis item, a score, and an alert.

The type of learning is an item included in the attribute data of the learning, and is an item for classifying the learning into a preset type. In the alert data 121 illustrated in FIG. 12, items of “online learning” and “online examination” are illustrated as types of learning. The type of learning may include, for example, specific subjects such as “mathematical online learning” and “English online examination”, but is not limited to the above items.

In the alert data 121 illustrated in FIG. 12, “the degree of concentration” and “the degree of empathy” are illustrated as analysis items corresponding to “online learning”. This indicates that, in the learning in which the type of learning is classified as the online examination, the alert is selected focusing on the “the degree of concentration” and the “degree of restlessness” among the analysis items included in the analysis data.

In the table illustrated in FIG. 12, scores “50-100” and scores “0-49” are illustrated on the right side of the “degree of concentration”. On the right side of each score, “Concentrating on” and “Degree of concentration is decreasing” are indicated as corresponding alerts. These indicate that “Concentrating on” can be selected as an alert when the score of “the degree of concentration” which is an analysis item is “50-100” in the type of learning “online class”. Similarly, in the type of learning “online class”, when the score of “the degree of concentration” which is an analysis item is “0-49”, it is indicated that “the degree of concentration is decreasing” can be selected as an alert.

In the table illustrated in FIG. 12, “the degree of empathy of a speaker” and “the degree of empathy of a non-speaker” are indicated as the analysis items of the “online class”. Furthermore, “0 40” is indicated in the score corresponding to “the degree of empathy of the speaker”, and “Compatibility with speaker seems to be poor” is further indicated as an alert. This suggests that the relationship between the speaker (for example, a teacher) and the student is bad. In the score corresponding to the “degree of empathy of the non-speaker”, “0-30” is indicated and “Compatibility with non-speakers seems to be poor” is further indicated as an alert. This suggests that the relationship between the students and the students around them is bad.

Furthermore, in the column under the type of learning “online class”, “online examination” is displayed. In the analysis item corresponding to the online examination, “the degree of concentration” and “the degree of restlessness” are shown. The score corresponding to the degree of concentration in the online examination is indicated as “0-40”, and the corresponding alert is indicated as “Degree of concentration seems to be low”. The score corresponding to the degree of restlessness is indicated as “80-100” and a corresponding alert is indicated as “cheating is suspicious”.

As described above, in the example illustrated in FIG. 12, the alert data 121 stores the type of learning, the analysis item, the score of the analysis item, and the alert in association with each other. The alert control unit 114 compares the learning data received from the learning data acquisition unit 112, the analysis data received from the analysis data generation unit 113, and the alert data 121 and selects a corresponding alert. Accordingly, the analysis apparatus 100 can supply the user with an alert appropriately selected in accordance with the learning attribute data, the score of the analysis data, and the like. The alert data 121 may adopt, for example, a subject of the learning, a purpose of learning, or the like, in addition to the type of learning, as the attribute data of learning.

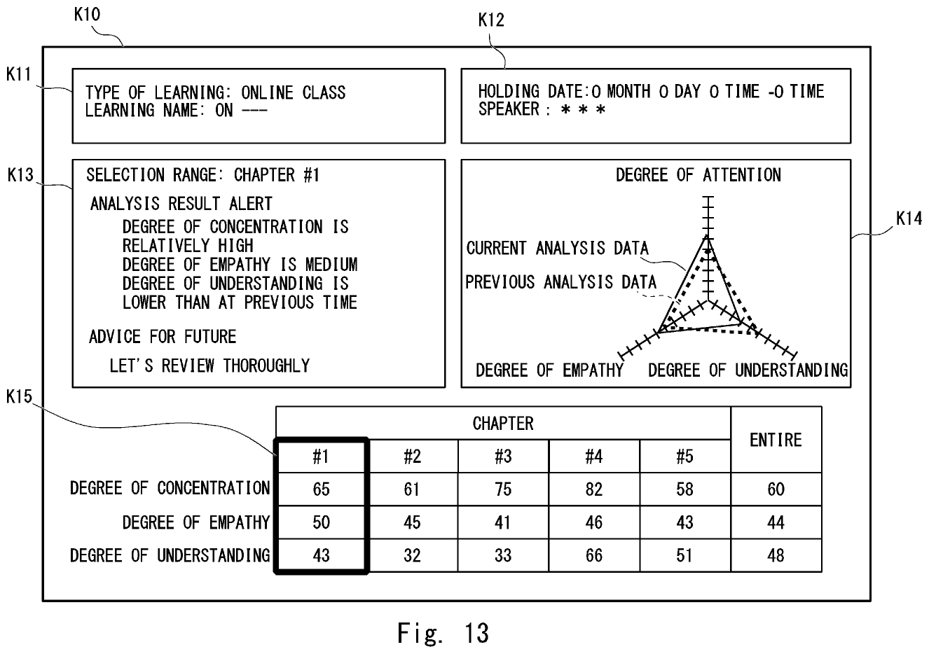

Next, an example of an analysis result will be described with reference to FIG. 13. FIG. 13 is a diagram illustrating a display example of an analysis result. FIG. 13 illustrates an analysis result K10 generated by the alert control unit 114. The analysis result K10 is a screen configured to be displayable on a display device of the manager terminal 990. The analysis result K10 includes a first display portion K11, a second display portion K12, a third display portion K13, a fourth display portion K14, and a fifth display portion K15.

In the first display portion K11, a type of learning and a learning name are displayed. In the second display portion K12, a date and time and a speaker of the learning are displayed. Data displayed on the first display portion K11 and the second display is included in learning data received from the learning data acquisition unit 112.

In the third display portion K13, an alert selected by the alert control unit 114 is displayed. In FIG. 13, “Selection range: chapter #1” is displayed in the third display portion K13. That is, the alert displayed in the third display portion K13 in FIG. 13 is an alert corresponding to chapter #1 in the learning. “Degree of concentration is relatively high”, “Degree of empathy is medium”, and “Degree of understanding is lower than at previous time” are displayed as “analysis result alert” in the third display portion K 13. Further, the third display portion K 13 displays “Let's review thoroughly” as “Advice for future”.

In the fourth display portion K14, analysis data in chapter #1 is shown as a radar chart. In the radar chart shown in the fourth display portion K14, the analysis data of chapter #1 is plotted by a solid line as “current analysis data”. In the radar chart shown on the fourth display portion K14, analysis data in the previous learning is plotted by a dotted line as “previous analysis data”. The previous analysis data is analysis data in a similar type of learning held in the past, and is data stored in the analysis history data 124. As illustrated in the drawing, the alert control unit 114 uses the analysis history data 124 to relatively compare the analysis data as a graph or a chart. Accordingly, the analysis apparatus 100 can show data that is easy to intuitively understand.

The fifth display portion K15 indicates analysis data of the entire learning and analysis data calculated for each chapter. In the fifth display portion K15, chapter #1 surrounded by a thick line indicates that the degree of concentration degree is 65, the degree of empathy is 50, and the degree of understanding is 43. These values correspond to the radar chart shown in the fourth display portion K14. These values correspond to the alert and the advice displayed in the third display portion K13.

When the analysis result K10 is displayed on the manager terminal 990, for example, when the user selects any chapter or an entire area of the fifth display portion K15, data corresponding to the selected area is displayed as the content of the third display portion K13 and the fourth display portion K14.

The example of the analysis result has been described above. The analysis apparatus 100 can generate alerts of various modes in addition to the above-described content. For example, the analysis apparatus 100 may express a tendency of the analysis data as a color tone and include an image with such a color tone in the alert.

Although the third example embodiment has been described above, the analysis system 10 according to the third example embodiment is not limited to the above-described configuration. For example, the analysis system 10 may include the learning management apparatus 400. In this case, the analysis apparatus 100, the emotion data generation apparatus 300, and the learning management apparatus 400 may be separate from each other, or some or all of the analysis apparatus 100, the emotion data generation apparatus 300, and the learning management apparatus 400 may be integrated. Furthermore, for example, the function of the emotion data generation apparatus 300 is configured as a program, and may be included in the analysis apparatus 100 or the learning management apparatus 400.

The above-described program can be stored and supplied to a computer using any of various types of non-transitory computer readable media. The non-transitory computer readable media include various types of tangible storage media. Examples of the non-transitory computer readable media include a magnetic recording medium (for example, a flexible disk, a magnetic tape, or a hard disk drive), a magneto-optical recording medium (for example, a magneto-optical disc), a compact disc-read only memory (CD-ROM), a CD-R, a CD-R/W, and a semiconductor memory (for example, a mask ROM, a programmable ROM (PROM), an erasable PROM (EPROM), a flash ROM, or a random access memory (RAM)). The program may be supplied to the computer using any of various types of transitory computer readable media. Examples of the transitory computer readable media include an electrical signal, an optical signal, and an electromagnetic wave. The transitory computer-readable media can supply programs to computers via a wired communication path such as electric wires and optical fibers, or wireless communication paths.

The present invention is not limited to the foregoing example embodiments, and can be appropriately changed without departing from the gist of the present invention. The plurality of examples described above can be implemented in appropriate combination.

Some or all of the foregoing example embodiments may be described as the following supplementary notes, but are not limited to the followings.

(Supplementary Note 1)

An analysis apparatus including:
an emotion data acquisition unit that acquires emotion data regarding learning of each learner, the emotion data being obtained by performing emotion analysis on face image data of a plurality of learners in online learning; and
an analysis data generation unit that aggregates emotion data regarding the plurality of learners, compares the emotion data of the plurality of learners, and generates analysis data in which the emotion data of one or more learners is identified based on a comparison result.

(Supplementary Note 2)

The analysis apparatus according to Supplementary note 1, further including:
a learning data acquisition unit that acquires learning data of the online learning,
wherein the analysis data generation unit generates analysis data by associating the emotion data of the one or more identified learners with the learning data.

(Supplementary Note 3)

The analysis apparatus according to Supplementary note 2, further including:
a chapter generation unit that generates a chapter at a predetermined switching timing of the learning data,
wherein the analysis data generation unit generates the analysis data by associating the emotion data of the one or more identified learners with the learning data for each generated chapter.

(Supplementary Note 4)

The analysis apparatus according to any one of Supplementary notes 1 to 3, wherein the analysis data generation unit calculates a distribution regarding a specific emotion from emotion data of the number of learners corresponding to at least one class, and identifies emotion data of one or more learners that is a deviation value based on the distribution.

(Supplementary Note 5)

The analysis apparatus according to any one of Supplementary notes 1 to 4, wherein the online learning includes an online class and an online examination, and the learner includes a student of the online class and an examinee of the online examination.

(Supplementary Note 6)

The analysis apparatus according to any one of Supplementary notes 1 to 5, further including:
an alert generation unit that notifies a terminal of a manager of the online learning or a terminal of the one or more learners identified based on a comparison result of the emotion data of the plurality of learners of an alert corresponding to the analysis data.

(Supplementary Note 7)

An analysis method including:
acquiring emotion data regarding learning of each learner, the emotion data being obtained by performing emotion analysis on face image data of a plurality of learners in online learning; and
aggregating emotion data regarding the plurality of learners, comparing the emotion data of the plurality of learners, and generating analysis data in which the emotion data of one or more learners is identified based on a comparison result.

(Supplementary Note 8)

An analysis program causing a computer to perform:
acquiring emotion data regarding learning of each learner, the emotion data being obtained by performing emotion analysis on face image data of a plurality of learners in online learning; and
aggregating emotion data regarding the plurality of learners, comparing the emotion data of the plurality of learners, and generating analysis data in which the emotion data of one or more learners is identified based on a comparison result.

Although the invention of the present application has been described above with reference to the example embodiments, the invention of the present application is not limited to the above. Various modifications that can be understood by those skilled in the art can be made to the configuration and details of the invention of the present application within the scope of the invention.

This application claims priority based on Japanese Patent Application No. 2021-029036 filed on Feb. 25, 2021, the disclosure of which is incorporated herein in its entirety.

### REFERENCE SIGNS LIST

10 ANALYSIS SYSTEM
90 LEARNING TERMINAL GROUP
100 ANALYSIS APPARATUS
111 EMOTION DATA ACQUISITION UNIT
112 LEARNING DATA ACQUISITION UNIT
113 ANALYSIS DATA GENERATION UNIT
114 ALERT CONTROL UNIT
115 OUTPUT UNIT
116 PERSON IDENTIFICATION UNIT
117 CHAPTER GENERATION UNIT
120 STORAGE UNIT
121 ALERT DATA
122 ANALYSIS RESULT STORAGE AREA
123 PERSON ATTRIBUTE DATA
124 ANALYSIS HISTORY DATA
300 EMOTION DATA GENERATION APPARATUS
311 LEARNER DATA ACQUISITION UNIT
312 EMOTION DATA GENERATION UNIT
313 EMOTION DATA OUTPUT UNIT
400 LEARNING MANAGEMENT APPARATUS
990 MANAGER TERMINAL
N NETWORK

What is claimed is:

1. An analysis apparatus comprising:
at least one memory storing instructions, and
at least one processor configured to execute the instructions to;
acquire emotion data regarding learning of each learner, the emotion data being obtained by performing emotion analysis on face image data of a plurality of learners in online learning; and
aggregate emotion data regarding the plurality of learners, comparing the emotion data of the plurality of learners, and generating analysis data in which the emotion data of one or more learners is identified based on a comparison result.

2. The analysis apparatus according to claim 1, wherein the at least one processor configured to execute the instructions to;
acquire learning data of the online learning, and
generate analysis data by associating the emotion data of the one or more identified learners with the learning data.

3. The analysis apparatus according to claim 2, wherein the at least one processor configured to execute the instructions to;
generate a chapter at a predetermined switching timing of the learning data, and
generate the analysis data by associating the emotion data of the one or more identified learners with the learning data for each generated chapter.

4. The analysis apparatus according to claim 1, wherein the at least one processor configured to execute the instructions to; calculate a distribution regarding a specific emotion from emotion data of the number of learners corresponding to at least one class, and identify emotion data of one or more learners that is a deviation value based on the distribution.

5. The analysis apparatus according to claim 1, wherein the online learning includes an online class and an online examination, and the learner includes a student of the online class and an examinee of the online examination.

6. The analysis apparatus according to claim 1, wherein the at least one processor configured to execute the instructions to;
notify a terminal of a manager of the online learning or a terminal of the one or more learners identified based on a comparison result of the emotion data of the plurality of learners of an alert corresponding to the analysis data.

7. An analysis method comprising:
acquiring emotion data regarding learning of each learner, the emotion data being obtained by performing emotion analysis on face image data of a plurality of learners in online learning; and
aggregating emotion data regarding the plurality of learners, comparing the emotion data of the plurality of learners, and generating analysis data in which the emotion data of one or more learners is identified based on a comparison result.

8. A non-transitory computer-readable medium storing an analysis program causing a computer to perform:
acquiring emotion data regarding learning of each learner, the emotion data being obtained by performing emotion analysis on face image data of a plurality of learners in online learning; and
aggregating emotion data regarding the plurality of learners, comparing the emotion data of the plurality of learners, and generating analysis data in which the emotion data of one or more learners is identified based on a comparison result.

9. The analysis method according to claim 7, further comprising:
acquiring learning data of the online learning, and
wherein the analysis data generation includes generating analysis data by associating the emotion data of the one or more identified learners with the learning data.

10. The analysis method according to claim 7, further comprising:
generating a chapter at a predetermined switching timing of the learning data, and
wherein the analysis data generation includes generating the analysis data by associating the emotion data of the one or more identified learners with the learning data for each generated chapter.

11. The analysis method according to claim 7, wherein the analysis data generation includes calculating a distribution regarding a specific emotion from emotion data of the number of learners corresponding to at least one class, and identifying emotion data of one or more learners that is a deviation value based on the distribution.

12. The analysis method according to claim 7, wherein the online learning includes an online class and an online examination, and the learner includes a student of the online class and an examinee of the online examination.

13. The analysis method according to claim 7, further comprising:
notifying a terminal of a manager of the online learning or a terminal of the one or more learners identified based on a comparison result of the emotion data of the plurality of learners of an alert corresponding to the analysis data.

14. The non-transitory computer-readable medium according to claim 8, further comprising:
acquiring learning data of the online learning, and
wherein the analysis data generation includes generating analysis data by associating the emotion data of the one or more identified learners with the learning data.

15. The non-transitory computer-readable medium according to claim 8, further comprising:
generating a chapter at a predetermined switching timing of the learning data, and
wherein the analysis data generation includes generating the analysis data by associating the emotion data of the one or more identified learners with the learning data for each generated chapter.

16. The non-transitory computer-readable medium according to claim 8, wherein the analysis data generation includes calculating a distribution regarding a specific emotion from emotion data of the number of learners corresponding to at least one class, and identifying emotion data of one or more learners that is a deviation value based on the distribution.

17. The non-transitory computer-readable medium according to claim 8, wherein the online learning includes an online class and an online examination, and the learner includes a student of the online class and an examinee of the online examination.

18. The non-transitory computer-readable medium according to claim 8, further comprising:
notifying a terminal of a manager of the online learning or a terminal of the one or more learners identified based on a comparison result of the emotion data of the plurality of learners of an alert corresponding to the analysis data.
